(19) United States
(12) Patent Application Publication
(10) Pub. No.: US 2026/0294695 A1
(43) Pub. Date: Oct. 1, 2026

(54) REUSABLE INCONTINENCE UNDERPANTS FOR MEN

(71) Applicant: SEAMPRINT GMBH & CO. KG, Münsing (DE)

(72) Inventors: Udo SIEBERT, Münsing (DE); Inge SIEBERT, Münsing (DE)

(73) Assignee: SEAMPRINT GMBH & CO. KG, Münsing (DE)

(21) Appl. No.: 19/489,072

(22) PCT Filed: May 29, 2024
(86) PCT No.: PCT/EP2024/064830

§ 371 Date: Dec. 1, 2025

(30) Foreign Application Priority Data
Jun. 1, 2023 (DE) 20 2023 103 023.8

(51) Int. Cl. A61F 13/471; A41B 9/02; A41B 9/12; A61F 5/453; A61F 13/491; A61F 13/505

(52) U.S. Cl. CPC A61F 13/471; A41B 9/023; A41B 9/12; A61F 5/453; A61F 13/4915; A61F 13/505

(57) ABSTRACT

Reusable incontinence underpants for men comprise a pocket for the penis which is arranged or formed in the crotch region on an inner side of the underpants. The penis pocket has an opening for receiving the penis which, relative to the orientation during wear, is arranged at the upper side of the penis pocket, is liquid-impermeable circumferentially and downwardly, relative to the orientation during wear, and contains a liquid-absorbent core arranged inside the penis pocket.

### FIELD OF THE INVENTION

The invention relates to reusable incontinence underpants for men comprising a pocket for the penis which is arranged or formed in the crotch region on the inner side of the underpants. The penis pocket has an opening for receiving the penis which, relative to the orientation during wear, is arranged at the upper side of the penis pocket. The penis pocket is liquid-impermeable circumferentially and downwardly, relative to the orientation during wear. A liquid-absorbent core is arranged inside the penis pocket.

### BACKGROUND OF THE INVENTION

Worldwide, 200 million people are affected by incontinence, i.e. the inability of the body to control urination, and the proportion of elderly people in society is expected to continue to increase. In 2022, sales for incontinence articles in Europe alone exceeded three billion euros.

Incontinence may be differentiated into mild, moderate and severe. Severe incontinence is characterised by sudden, uncontrolled bladder emptying with urine volumes greater than 200 mL/4 h. For severe incontinence, aids with the highest absorbent strength, such as disposable pants or diaper pants, are recommended. Diaper pants in particular provide good leakage protection.

However, diaper pants are embarrassing for many sufferers. They are unaesthetic and identify the wearer at first glance. In addition, rising environmental awareness creates a desire to use reusable alternatives instead of disposable articles. It is therefore an object of the present invention to provide an alternative to diaper pants that is more appealing and helps reduce waste volume. This object is achieved by the invention.

### DESCRIPTION OF THE INVENTION

A first aspect of the invention relates to reusable incontinence underpants for men comprising a penis pocket arranged or formed in the crotch region on the inner side of the underpants. The penis pocket has an opening for receiving the penis which, relative to the orientation during wear, is arranged at the upper side of the penis pocket. The penis pocket is liquid-impermeable circumferentially and downwardly, relative to the orientation during wear, and a liquid-absorbent core (also referred to herein as an absorbent core) is arranged inside the penis pocket.

The reusable incontinence underpants are particularly distinguished by an aesthetic appearance in the form of ordinary men's underpants. A further advantage is their reusability: they are washable at 90° C. and can be dried.

In a preferred embodiment, the penis pocket comprises a liquid-impermeable first layer which is closest to the body. The first layer may comprise a textile sheet coated with an adhesive coating material. The coating material may be or comprise an elastomer, preferably silicone. Preferably, the elastomer, in particular the silicone, is printed onto the textile. Other modes of application are also possible. The coating may be applied on the body-facing or the body-remote side of the textile sheet. In addition, the coating may be flocked. The first layer may also comprise a mesh layer. The mesh layer or the textile sheet may be the layer closest to the body (when the underpants are worn).

A further preferred embodiment of the penis pocket provides a second layer that is liquid-absorbent and/or liquid-transporting and which, together with the first layer, forms the opening for receiving the penis. The second layer may comprise a mesh layer and a liquid-absorbent layer. Preferably, the mesh layer is closer to the body than the liquid-absorbent layer. The liquid-absorbent layer of the first layer may be based on terry, non-woven, molleton or polyester, may comprise terry, non-woven, molleton or polyester, or may consist thereof.

Preferred penis pockets further comprise a liquid-impermeable third layer which, together with the first layer, seals the penis pocket circumferentially and downwardly, relative to the orientation during wear. The third layer may comprise a textile sheet coated with an adhesive coating material, preferably as defined above for the first layer. The coating may be applied on the body-facing or the body-remote side of the textile sheet. Preferably, the coating is flocked. The third layer may further comprise a mesh layer, the mesh layer being closer to the body than the textile sheet, or vice versa.

It is furthermore preferred that the third layer comprises a liquid-absorbent layer. The liquid-absorbent layer of the third layer may be closer to the body than the textile sheet of the third layer. Preferably, the liquid-absorbent layer of the third layer is based on terry, non-woven, molleton or polyester, or comprises or consists of terry, non-woven, molleton or polyester.

A replaceable and/or permanent liquid-absorbent core may be arranged between the second and third layers. In particular, the second layer together with the third layer may form an upwardly open pocket for inserting (and removing) a (replaceable) liquid-absorbent core. In the case of a replaceable absorbent core, it is preferably based on, comprises or consists of a superabsorbent polymer.

In a further preferred embodiment, a fourth layer adjoins the body-remote side of the third layer. The fourth layer may comprise a textile sheet. The fourth layer may form the outermost layer of the underpants.

To ensure tightness at adjoining layer edges, various measures are possible. By way of example: where the penis pocket is formed by two liquid-impermeable layers, for example the first and third layers as defined above, the two layers may be sewn together at their lateral and lower edge regions and, where appropriate, sealed by means of a sealing tape.

Alternatively, where the penis pocket is formed by two liquid-impermeable layers, preferably the first and third layers as defined above, the two layers may be sealed at their lateral and lower edge regions by folding over and sewing and, where appropriate, sealing the needle holes by means of a melt film.

In a further alternative, the penis pocket may be formed from a bag-shaped liquid-impermeable layer.

The various layers of the layer structure described herein may be interconnected by joints selected from seam joints, adhesive joints and welded joints, and combinations thereof, in particular combinations of seam joints and adhesive joints or combinations of seam joints and welded joints.

Within the scope of the present invention, the particular configuration of the penis pocket may vary depending on requirements. The position of the opening of the penis pocket may be adapted to the male anatomy. Furthermore, the circumference and/or the length of the penis pocket may be adapted to the male anatomy. The circumference and/or length of the penis pocket and/or the circumference and/or length of the pocket for inserting the replaceable absorbent core may be adapted to the volume of liquid to be taken up (i.e. the severity of incontinence). In this connection, it is envisaged that the penis pocket, and in particular the part forming the pocket for inserting the absorbent core, extends into the rear (i.e. buttocks) region of the underpants.

In addition, the opening of the penis pocket may be at the same height as, or lower than, the opening of the pocket for inserting the absorbent core. The penis pocket may moreover comprise, adjacent to the opening, a slit or a cut-out in the layer closest to the body, preferably in the first layer as defined above. Alternatively, the layer of the penis pocket that is closest to the body, preferably the first layer as defined above, may be formed by two partially overlapping sections. It is also possible for the penis pocket to be made of an elastic material.

The opening of the pocket for inserting the (replaceable) absorbent core is accessible from the inner side of the underpants. The replaceable absorbent core may be reusable (i.e. washable) or a disposable article.

A further aspect of the invention relates to the medical or non-medical use of incontinence underpants as defined herein for mild, moderate or severe incontinence.

A further aspect of the invention relates to the manufacture of incontinence underpants as defined herein, comprising:
providing an underpants base;
producing the penis pocket;
fastening the penis pocket in the crotch region of the underpants base; and,
optionally, applying a sealing tape in the region where the penis pocket has been fastened in the crotch region.

Finally, it is noted that any feature described with respect to a specific aspect or embodiment is intended to be combinable with other aspects or embodiments, unless the combination is technically nonsensical or impossible.

### BRIEF DESCRIPTION OF THE DRAWINGS

The inventive concept, non-limiting embodiments and further advantages of the concept will now be described with reference to the drawings, in which:

FIG. 1 shows a turned-inside-out front view of men's incontinence underpants with a penis pocket according to the invention.

FIG. 2 shows various configurations of penis pockets.

FIG. 3 shows the front (left) and rear (right) sides of turned-inside-out underpants with a penis pocket according to the invention.

FIG. 4 shows a preferred layer structure of a penis pocket in cross-section.

### DETAILED DESCRIPTION OF THE DRAWINGS

The description of the invention is given here by way of example only and is in no way to be understood as limiting the scope or extent of the invention.

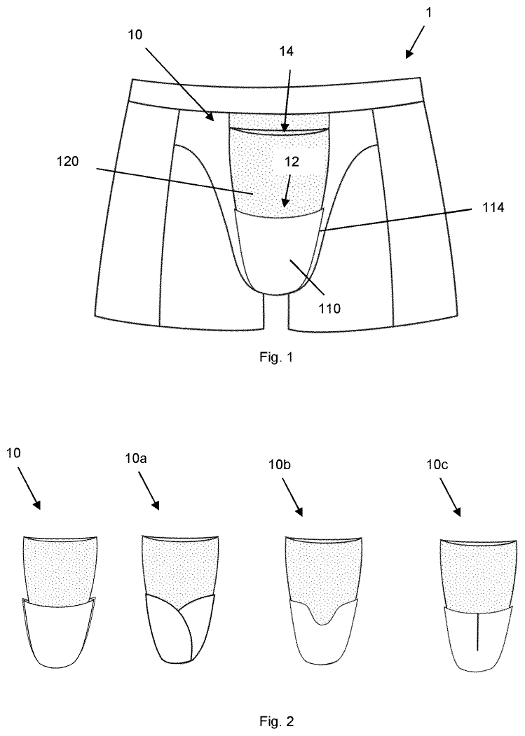

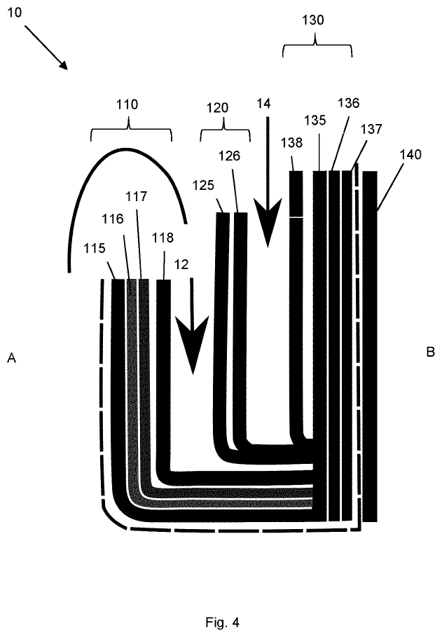

FIG. 1 shows the front of turned-inside-out men's incontinence underpants (1) according to the invention with a penis pocket (10) arranged in the crotch region on the inner side of the underpants (1). The penis pocket (10) has an opening (12) for receiving the penis which, relative to the orientation during wear, is arranged at the upper side of the penis pocket (10). The penis pocket (10) is liquid-impermeable circumferentially and downwardly, relative to the orientation during wear (along edge 114). The penis pocket (10) has a liquid-impermeable first layer (110) which is closest to the body. The penis pocket (10) has a first layer (110) and a second layer (120) which are described in more detail in connection with FIG. 4. FIG. 1 also shows an opening (14) for inserting a replaceable absorbent core into the interior of the penis pocket (10).

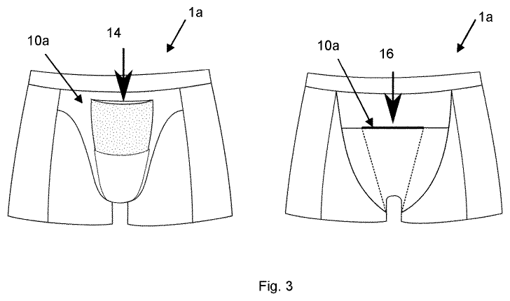

FIG. 2 illustrates various configurations of the penis pocket (10, 10a, 10b, 10c) in inside view. Compared with the penis pocket (10) shown in FIG. 1, the penis pocket (10a) is formed by two partially overlapping sections. Penis pocket (10b) has a cut-out in the layer that is closest to the body. Penis pocket (10c) has a slit in the layer that is closest to the body.

FIG. 3 shows the front (left) and rear (right) sides of turned-inside-out underpants (1a) according to the invention with a penis pocket (10a). In this example, the length of the pocket for inserting the replaceable absorbent core extends into the rear (i.e. buttocks) region of the underpants. This allows use of an absorbent core with higher liquid uptake capacity. The arrow in the right illustration of FIG. 2 indicates that the absorbent core, or a further absorbent core, can be inserted from the rear through an opening (16). The opening (16) may be provided in addition to, or instead of, the opening (14).

FIG. 4 illustrates a preferred layer structure of a penis pocket (10) in cross-section. From the inner side A (body side) towards the outer side B, the penis pocket (10) comprises three layers (110, 120 and 130) which form the penis pocket (10). A fourth layer (140) forms the outermost layer. The layers are described below.

The first layer (110) is liquid-impermeable and directly adjoins the body of the wearer. It is multilayered and comprises a textile sheet (115) provided with a liquid-impermeable coating (116). The coating (116) is located on the body-remote side of the textile sheet (115). Furthermore, on its body-remote side the coating (116) has a flocking layer (117). The first layer (110) further comprises a mesh layer (118) which forms an inner side of the penis pocket (10).

The second layer (120) is liquid-absorbent and/or liquid-transporting. Together with the first layer (110) it forms the opening (112) for receiving the penis. The second layer (120) comprises a mesh layer (125) and a liquid-absorbent and/or liquid-transporting layer (126). Preferably, the mesh layer (125) is closer to the body than the layer (126).

The third layer (130) is liquid-impermeable. Together with the first layer (110) it seals the penis pocket (10) circumferentially and downwardly, relative to the orientation during wear. The third layer (130) comprises a textile sheet (135). The textile sheet (135) is coated with a liquid-impermeable coating (136). The coating (136) is located on the body-remote side of the textile sheet (135). Furthermore, the coating (136) has a flocking layer (137). The third layer (130) further comprises a layer (138) (a mesh layer and/or a liquid-absorbent layer) which is closer to the body than the textile sheet (135).

A replaceable and/or permanent absorbent core may be arranged between the second layer (120) and the third layer (130). In particular, the second layer (120) together with the third layer (130) forms an upwardly open pocket for inserting an absorbent core. The absorbent core can be inserted through the opening (14).

A fourth layer (140) adjoins the body-remote side of the third layer (130). The fourth layer (140) may comprise a textile sheet. The fourth layer (140) may form the outermost layer of the underpants (1). In the embodiment shown, the fourth layer (140) is single-layered.

1. A reusable incontinence underpants for men comprising a pocket for the penis which is arranged or formed in the crotch region on the inner side of the underpants, wherein the penis pocket has an opening for receiving the penis which, relative to the orientation during wear, is arranged at the upper side of the penis pocket, wherein the penis pocket is liquid-impermeable circumferentially and downwardly, relative to the orientation during wear, and wherein a liquid-absorbent core is arranged inside the penis pocket.

2. The underpants according to claim 1, wherein the penis pocket comprises a liquid-impermeable first layer.

3. The underpants according to claim 2, wherein the first layer comprises a textile sheet which is coated with an adhesive coating material, the coating material being or comprising an elastomer.

4. The underpants according to claim 3, wherein the elastomer is printed on the body-facing or on the body-remote side of the textile sheet.

5. The underpants according to claim 2, wherein the first layer comprises a mesh layer, and the mesh layer is the layer closest to the body or the textile sheet is the layer closest to the body.

6. The underpants according to claim 2, wherein the penis pocket comprises a second layer which is liquid-absorbent and/or liquid-transporting and which, together with the first layer, forms the opening for receiving the penis.

7. The underpants according to claim 6, wherein the second layer comprises a mesh layer and a liquid-absorbent layer, and the mesh layer is closer to the body than the liquid-absorbent layer.

8. The underpants according to claim 2, wherein the penis pocket comprises a liquid-impermeable third layer which, together with the first layer, seals the penis pocket circumferentially and downwardly relative to the orientation during of wear.

9. The underpants according to claim 8, wherein the third layer comprises a textile sheet coated with an adhesive coating material, the coating material being or comprising an elastomer.

10. The underpants according to claim 8, wherein the third layer comprises a liquid-absorbent layer, the liquid-absorbent layer of the third layer being closer to the body than the textile sheet of the third layer.

11. The underpants according to claim 8, wherein one or more of the following is fulfilled:
the second layer together with the third layer forms an upwardly open pocket for inserting a replaceable liquid-absorbent core;
a replaceable liquid-absorbent core is arranged between the second and third layers;
a permanent liquid-absorbent core is arranged between the second and third layers; and
the liquid-absorbent core comprises a superabsorbent polymer.

12. The underpants according to claim 1, wherein the penis pocket is formed from:
(a) two liquid-impermeable layers which are sewn together at the lateral and lower edge regions of the two layers and are sealed by means of a sealing tape; or
(b) from two liquid-impermeable layers which are sealed at the lateral and lower edge regions of the two layers by folding over and sewing and by sealing the needle holes by means of a melt film; or
(c) from a bag-shaped liquid-impermeable layer.

13. The underpants according to claim 1, wherein one or more of the following is fulfilled:
the position of the opening of the penis pocket is adapted to male anatomy;
the circumference and the length of the penis pocket are adapted to the male anatomy;
the circumference and the length of the penis pocket are adapted to the volume of liquid to be taken up; and
the circumference and the length of the pocket for inserting the replaceable liquid-absorbent core are adapted to the volume of liquid to be taken up;
the penis pocket extends into the rear region of the underpants;
the pocket for inserting the liquid-absorbent core extends into the rear region of the underpants.

14. A method for treating incontinence comprising wearing the underpants of claim 1.

15. A method for producing the underpants as defined in claim 1, the method comprising:
providing an underpants base;
producing a penis pocket which is liquid-impermeable circumferentially and downwardly relative to the orientation during wear; and
fastening the penis pocket in the crotch region of the underpants base.

16. The underpants according to claim 4, wherein the first layer comprises a mesh layer, and the mesh layer is the layer closest to the body or the textile sheet is the layer closest to the body.

17. The underpants according to claim 16, wherein the penis pocket comprises a second layer which is liquid-absorbent and/or liquid-transporting and which, together with the first layer, forms the opening for receiving the penis.

18. The underpants according to claim 9, wherein the third layer comprises a liquid-absorbent layer, the liquid-absorbent layer of the third layer being closer to the body than the textile sheet of the third layer.

19. The underpants according to claim 18, wherein one or more of the following is fulfilled:
the second layer together with the third layer forms an upwardly open pocket for inserting a replaceable liquid-absorbent core;
a replaceable liquid-absorbent core is arranged between the second and third layers;
a permanent liquid-absorbent core is arranged between the second and third layers; and
the liquid-absorbent core comprises a superabsorbent polymer.

20. The underpants according to claim 19, wherein the penis pocket is formed from:
(a) two liquid-impermeable layers which are sewn together at the lateral and lower edge regions of the two layers and are sealed by means of a sealing tape; or
(b) from two liquid-impermeable layers which are sealed at the lateral and lower edge regions of the two layers by folding over and sewing and by sealing the needle holes by means of a melt film; or
(c) from a bag-shaped liquid-impermeable layer.
